(19) United States
(12) Patent Application Publication
(10) Pub. No.: US 2017/0304042 A1
(43) Pub. Date: Oct. 26, 2017

(54) STENT GRAFT HAVING EXTENDED LANDING AREA AND METHOD FOR USING THE SAME

(71) Applicant: ST. JUDE MEDICAL, CARDIOLOGY DIVISION, INC., St. Paul, MN (US)

(72) Inventor: Brooke Ren, Maple Grove, MN (US)

(21) Appl. No.: 15/643,659

(22) Filed: Jul. 7, 2017

Related U.S. Application Data
(63) Continuation of application No. 12/197,604, filed on Aug. 25, 2008, now abandoned.

(51) Int. Cl. A61F 2/07; A61F 2/856; A61F 2/90; A61F 2/06; A61F 2/82

(52) U.S. Cl. CPC A61F 2/07; A61F 2/856; A61F 2220/0058; A61F 2220/005; A61F 2210/0076; A61F 2210/0019; A61F 2002/825; A61F 2002/061; A61F 2/90; A61F 2250/0007; A61F 2210/0028

(57) ABSTRACT

A medical device for treating a target site within a lumen having an arcuate portion is provided. The medical device includes a first tubular portion comprising a proximal and distal end, and a second tubular portion comprising a proximal and distal end. A linking portion couples the first and second tubular portions, and an opening defined between the distal end of the first tubular portion and the proximal end of the second tubular portion. At least part of the linking portion is configured to conform to at least a portion of the arcuate portion of the lumen. Associated methods for using a medical device are also provided.

### FIELD OF THE INVENTION

The present invention relates to medical devices and associated methods for treating various target sites and, in particular, to medical devices configured for use in arcuate lumens and associated methods for delivering such medical devices.

### BACKGROUND OF THE INVENTION

An aortic aneurysm is a weak area in the aorta wall, which may be caused, for example, by arteriosclerosis. As blood flows through the aorta, the weak area of the vessel wall thins over time and expands like a balloon. Most commonly, aortic aneurysms occur in the portion of the vessel below the renal artery origins.

Eventually, an untreated aortic aneurysm will burst if the vessel wall gets too thin. Such rupturing of an aortic aneurysm frequently leads to death. As such, once an aneurysm reaches about 5 cm in diameter, it is usually considered necessary to treat to prevent rupture (below 5 cm, the risk of the aneurysm rupturing is considered lower than the risk of conventional heart surgery in patients with normal surgical risks).

Aneurysms, including aortic aneurysms, may be treated with surgery. The surgical procedure for treating an aortic aneurysm involves replacing the affected portion of the aorta with a synthetic graft, usually comprising a tube made out of an elastic material with properties very similar to that of a normal, healthy aorta. However, surgical treatment is complex and may pose additional risks to the patient, especially the elderly.

More recently, instead of performing surgery to repair an aortic aneurysm, vascular surgeons have installed an endovascular stent graft delivered to the site of the aneurysm using elongated catheters. An endovascular stent graft is a tube composed of blood impervious fabric supported by a metal mesh called a stent. It can be used for a variety of conditions involving the blood vessels, but most commonly is used to reinforce aneurysms. Typically, the surgeon will make a small incision in the patient's groin area and then insert into the vasculature a delivery catheter containing a collapsed, self-expanding or balloon-expandable stent graft. The delivery catheter is advanced to a location bridging the aneurysm, at which point the stent graft is delivered out from the delivery catheter and expanded to approximately the normal diameter of the aorta at that location. Over time, the stent graft becomes endothelialized and the space between the outer wall of the stent graft and the aneurysm ultimately fills with clotted blood, which prevents the aneurysm from growing further.

Depending on where the location of the aneurysm is within a vessel relative to other branch vessels, different design variations of the stent graft may be needed. For example, in treating an aortic aneurysm in the area of the renal arteries, the stent graft should be placed so as not to exclude blood flow through the renal arteries. Moreover, the stent graft should be anchored within the lumen, such as by promoting endothelialization or fixation with the lumen, in order to reduce the incidence of migration. Enhanced fixation of the stent graft to the arterial wall may also reduce the occurrence of endoleaks or blood flowing around the stent, which may prevent further weakening of the arterial wall at the site of the aneurysm.

Providing for adequate fixation of a stent graft in the area of the aortic arch can be challenging due to the various arteries that branch from the aorta in that region. The stent graft must provide adequate contact force against the vessel walls to prevent migration and endoleaks, but must not restrict blood flow to the branching arteries.

Therefore, there is a need for a stent graft that is capable of being deployed in a lumen having an arcuate portion, such as in the vicinity of the aortic arch. The stent graft should easily be deliverable and should be capable of being adequately anchored within the lumen.

### SUMMARY OF THE INVENTION

Embodiments of the present invention provide a medical device, such as, for example, a stent graft, for treating a target site within the body. For example, one embodiment provides a medical device for treating a target site within a lumen having an arcuate portion and at least one branch lumen extending therefrom. The medical device includes a first tubular portion comprising a proximal and distal end, and a second tubular portion comprising a proximal and distal end. The first and second tubular portions each may include at least one layer of a metallic material, such as a shape memory alloy, that is configured to be heat set to an expanded heat set configuration, and in some cases may include multiple layers. The first and second tubular portions may each be configured to be constrained to a smaller diameter than the respective expanded heat set configuration, for example, for delivery within a catheter, and may return to the respective expanded heat set configuration when deployed from the catheter.

The medical device further includes a linking portion that may be, for example, a filament, a fiber, a wire, a cord, a cable, a braid, a fabric, and/or a beam, that couples the first and second tubular portions. At least part of the linking portion may have a preset, memorized arcuate configuration that is configured to conform to at least a portion of the arcuate portion of the lumen. The linking portion may be resilient and/or adjustable in length. An opening may be defined between the distal end of the first tubular portion and the proximal end of the second tubular portion and be configured to align with the at least one branch lumen and facilitate fluid flow between the at least one branch lumen and the arcuate portion of the lumen. According to one aspect, a location of each of the linking portion and the opening within the arcuate portion of the lumen may be rotationally dependent on a location of the at least one branch lumen.

The first tubular portion may be, for example, a stent graft configured to be positioned downstream of the arcuate portion of the lumen, such as within a descending thoracic aorta. The second tubular portion may be configured to anchor the medical device upstream of the arcuate portion of the lumen, such as within an ascending thoracic aorta. The linking portion may be configured to be positioned within the arcuate portion of the lumen, such as within an aortic arch, such that the opening defined between the first and second tubular portions is configured to align with at least one branch lumen extending from the arcuate portion of the lumen. In some embodiments, the first tubular portion, said second tubular portion, and linking portion can be integrally formed from a common material, such as a braided metallic material.

In another embodiment, a medical device for treating an aneurysm within an aortic arch is provided. The medical device includes a first tubular portion configured to be positioned within a descending thoracic aorta and a second tubular portion configured to be positioned within an ascending thoracic aorta. The medical device further includes a linking portion coupling the first and second tubular portions and an opening defined between the first and second tubular portions. The linking portion is configured to be positioned within, and conform at least partially to, the aortic arch. The opening is configured to align with at least one artery extending from the aortic arch.

In yet another embodiment, a method of delivering a medical device to a target site within a lumen having an arcuate portion and at least one branch lumen extending therefrom is provided. The method includes providing a medical device that has a first tubular portion comprising a proximal and distal end, a second tubular portion comprising a proximal and distal end, and a linking portion coupling the first and second tubular portions. The medical device also includes an opening defined between the distal end of the first tubular portion and the proximal end of the second tubular portion. The first and second tubular portions and the linking portion can be constrained from respective expanded configurations to a smaller diameter for delivery within a catheter, for example, by respectively axially elongating the tubular and linking portions. The medical device can be delivered, for example, over a guidewire, to the target site, where the device can be deployed from the catheter such that the first and second tubular portions respectively assume their expanded configurations, the linking portion conforms to the arcuate portion of the lumen, and the opening aligns with the at least one branch lumen and facilitates fluid flow between the at least one branch lumen and the arcuate portion of the lumen.

In some embodiments, the first and second tubular portions respectively self-expand and return to their expanded configurations when deployed from the catheter. In other embodiments, the first and second tubular portions may be axially compressed so as to urge the first and second tubular portions to return to the respective expanded configurations. In some embodiments, the medical device can be deployed from the catheter such that the second tubular portion is disposed within the ascending thoracic aorta of the body, the first tubular portion is disposed within the descending thoracic aorta of the body, and the linking portion is disposed within, and conforms generally to, at least a portion of the shape of the aortic arch. In addition, the deployment of the medical device may be rotationally dependent on a location of each of the linking portion and the opening within the arcuate portion of the lumen with respect to a location of the at least one branch lumen.

### BRIEF DESCRIPTION OF THE DRAWINGS

Having thus described the invention in general terms, reference will now be made to the accompanying drawings, which are not necessarily drawn to scale, and wherein:

FIG. 1 is a perspective view of a medical device configured in accordance with an exemplary embodiment;

FIG. 2 is a perspective view of the medical device of FIG. 1 in a constrained configuration;

FIG. 3 is a cross-sectional view of the medical device of FIG. 1 deployed at a target site around an aortic arch;

FIGS. 4-6 are perspective views demonstrating a process for fabricating a medical device in accordance with an exemplary embodiment;

FIG. 7 is a side view of the medical device of FIG. 1 disposed within the bore of a delivery catheter;

FIG. 8 is a perspective view of a medical device configured in accordance with another exemplary embodiment;

FIG. 9 is a cross-sectional view of the medical device of FIG. 8 taken along line 9-9 of FIG. 8;

FIGS. 10 and 11 are perspective views of medical devices configured in accordance with still other exemplary embodiments;

FIG. 12 is an exploded perspective view of the medical device of FIG. 10;

FIGS. 13A-B are perspective views demonstrating a process for fabricating a medical device according to an additional embodiment;

FIG. 14 is a perspective view of a medical device according to another embodiment of the present invention; and

FIGS. 15-17 are perspective views of medical devices having an adjustable linking portion according to various embodiments of the present invention.

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

Embodiments of the present invention provide a medical device for use in treating a target site within the body, such as excluding or occluding various vascular abnormalities, which may include, for example, excluding an aneurysm. The device may also be used as a graft for lining a lumen of a vessel. It is understood that the use of the term “target site” is not meant to be limiting, as the device may be configured to treat any target site, such as an abnormality, a vessel, an organ, an opening, a chamber, a channel, a hole, a cavity, or the like, located anywhere in the body. For example, the abnormality could be any abnormality that affects the shape or the function of the native lumen, such as an aneurysm, a lesion, a vessel dissection, flow abnormality or a tumor. Furthermore, the term “lumen” is also not meant to be limiting, as the abnormality may reside in a variety of locations within the vasculature, such as a vessel, an artery, a vein, a passageway, an organ, a cavity, or the like.

As used herein the term “proximal” shall mean closest to the operator (less into the body) and “distal” shall mean furthest from the operator (further into the body). In positioning of the medical device from a downstream access point, distal is more upstream and proximal is more downstream.

As explained in further detail below, embodiments of the present invention provide medical devices for treating various target sites. The medical devices may include tubular portions separated and coupled by an arcuate or flexible linking portion. The arcuate or flexible linking portion may allow the medical device to conform to an arcuate target site. Moreover, the arcuate or flexible linking portion may enable the tubular portions to be disposed on opposing sides of an arcuate portion of a target site, which may improve the fixation of the medical device at such an arcuate target site. Furthermore, the medical device may include an opening configured to align with one or more branch lumens extending from the arcuate portion in order to reduce blockage of the one or more branch lumens.

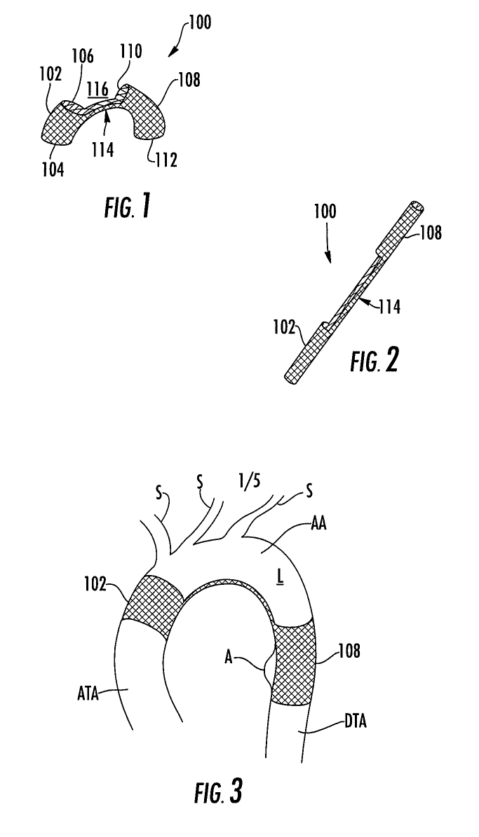

Referring to FIG. 1, therein is shown a medical device 100 configured in accordance with an exemplary embodiment. The medical device 100 includes a first tubular portion, such as a stent graft 108, which has a proximal 112 end and a distal end 110. The medical device 100 also includes a second tubular portion, such as an anchoring structure 102, having a proximal end 106 and a distal end 104. Each of the first and second tubular portions may be generally cylindrical, but could be various shapes depending on the configuration of the lumen in which the tubular portions are to be positioned. An arcuate/linking portion 114 couples the stent graft 108 and anchoring structure 102, and an opening 116 extending between the proximal end 106 of the anchoring structure 102 and the distal end 110 of the stent graft 108.

Referring to FIGS. 1 and 2, one or both of the stent graft 108 and anchoring structure 102 may include at least one layer of a metallic material, the layer being in the form of, for example, a sheet or a woven, knitted, or braided tubular metallic fabric. The fabric can be composed of multiple metallic strands. Although the term “strand” is discussed herein, “strand” is not meant to be limiting, as it is understood the fabric may comprise one or more wires, cords, fibers, yarns, filaments, cables, threads, or the like, such that such terms may be used interchangeably. The stent graft 108 and anchoring structure 102 may be a variety of occlusive materials capable of at least partially inhibiting blood flow therethrough in order to facilitate the formation of thrombus and epithelialization around the device. Moreover, the stent graft 108 and anchoring structure 102 could be a self-expandable or balloon-expandable material, such as stainless steel or etched stents. For example, the stent graft 108 and anchoring structure 102 could be independently expanded via respective balloons. One could mount the anchoring structure 102 over the balloon of a balloon catheter and the balloon after inflating and expanding the anchoring structure could be deflated and retracted into the stent graft and used to expand the stent graft. Alternatively the anchoring structure and the stent graft could be mounted on a catheter having two spaced apart balloons where by each of the anchoring and the stent graft portions are mounted respectively onto the distal and proximal balloons such as by crimping.

In some embodiments, one or both of the stent graft 108 and anchoring structure 102 may include multiple layers of metallic material. The layers may have different porosity or opening sizes. In addition, one layer may be for structural support and a second layer may inhibit blood flow through the layer. The layer(s) of a metallic material can be configured to be heat set to an expanded heat set configuration. For example, in one embodiment, one or both of the stent graft 108 and anchoring structure 102 can be composed at least partially of a shape memory material in order to provide for being heat set in an expanded configuration and to retain the expanded shape at or below body temperature. The stent graft 108 and anchoring structure 102 can then be configured to be constrained to a smaller diameter than their respective expanded heat set configurations for delivery through a catheter to a target location within the body. For example, the stent graft 108 and anchoring structure 102 may be braided tubular structures that have expanded configurations in which the outer diameters of the stent graft and anchoring structure are approximately the same as the inner diameter of the aorta, and which can be reduced to smaller diameters for delivery within a catheter, such as by axially elongating the stent graft and anchoring structure. The linking portion may be configured to pass through a catheter without necessarily needing any axial elongation.

In one embodiment, the stent graft 108, anchoring structure 102, and/or linking portion 114 are formed from a shape memory alloy, such as Nitinol. It is also understood that the stent graft 108, anchoring structure 102, and/or linking portion 114 may comprise various materials other than Nitinol that have highly elastic properties, such as spring stainless steel, and alloys such as Elgiloy®, Hastelloy®, CoCrNi alloys (e.g., trade name Phynox), MP35N®, or CoCrMo alloys.

According to one embodiment, each layer of the device may comprise 36-144wire strands ranging in diameter from about 0.0005 to 0.010 in. formed of a shape memory alloy that are braided so as to define fenestrations with an area of about 0.00015 to 0.0015 sq. in., which are sufficiently small so as to slow the blood flow through the wall of the device and to facilitate thrombus formation thereon. Inner and outer braided layers may have pitch angles that are about equal to obtain desirable collapse and expansion characteristics, such as maintaining a uniform overall length. The stiffness of the device may be increased or decreased by altering the wire strand size, the shield angle, the pick rate, and the number of wire strand carriers or the heat treatment process.

Thus, the stent graft 108 can also be configured to facilitate thrombosis, for example, by at least partially inhibiting blood flow therethrough in order to facilitate the formation of thrombus and epithelialization around the stent graft. In particular, the braid of a metallic fabric may be chosen to have a predetermined pick and pitch to define openings or fenestrations so as to vary the impedance of blood flow therethrough. For instance, the formation of thrombus may result from substantially precluding or impeding flow, or functionally, that blood flow may occur for a short time, e.g., about 3-60 minutes through the metallic fabric, but that the body's clotting mechanism or protein or other body deposits on the braided wire strands results in occlusion or flow stoppage after this initial time period. For instance, occlusion may be clinically represented by injecting a contrast media into the upstream lumen of the stent graft 108 and if no contrast media flows through the wall of the stent graft after a predetermined period of time as viewed by fluoroscopy, then the position and occlusion of the stent graft is adequate. Moreover, occlusion of the target site could be assessed using various ultrasound echo doppler modalities. Although the stent graft 108 has been described as having one or more layers of occlusive material, it is understood that the anchoring structure 102 and/or linking portion 114 may also or alternatively include one or more layers of occlusive material to facilitate thrombosis or the wires may be coated with a thrombus promoting substance.

According to one embodiment, the stent graft 108 could be configured to be positioned within a lumen having an aneurysm. For instance, the stent graft 108 could be positioned within a lumen having an aneurysm A in the descending thoracic aorta (DTA). In addition or alternatively, the anchoring structure 102 could comprise occlusive material and be configured to exclude an aneurysm in the ascending thoracic aorta (ATA).

The linking portion 114 may have a preset, memorized arcuate configuration or be flexible enough to easily conform to the curvature of an arcuate portion of a lumen. In some embodiments, the arcuate configuration may conform to at least a portion of an arcuate portion of the lumen of a vessel, this aspect making such embodiments well-suited for deployment within a lumen having an arcuate portion, such as, for example, the aortic arch (AA). For example, referring to FIG. 3, the stent graft 108 can be configured to be positioned downstream of the arcuate portion of the lumen L (e.g., in the DTA), the anchoring structure 102 can be configured to anchor the medical device upstream of the arcuate portion of the lumen (e.g., in the ATA), and the linking portion 114 can be configured to be positioned within the arcuate portion of the lumen (e.g., in the AA). In this way, the opening 116 defined between the stent graft 102 and the anchoring structure 108 may be configured to align with at least one side or branch lumen S extending from the arcuate portion of the lumen L. Thus, the linking portion 114 may be configured to conform to the arcuate portion of the lumen opposite the branch lumens S, while the opening 116 is configured to facilitate fluid flow between the arcuate portion and the branch lumens. Thus, the location of the linking portion 114 and the opening 116 may be rotationally dependent on one another.

The linking portion 114 can include a filament, a fiber, a wire, a cord, a cable, a braid, a fabric, and/or a beam. The linking portion 114 can be composed at least partially of a shape memory material and may be heat set in the arcuate configuration. Alternatively, the linking portion 114 can be formed (e.g., molded or cold-worked) so as to have an arcuate shape and highly elastic properties so as to pass through a catheter and retain its arcuate shape upon exiting the catheter. Moreover, the stent graft 102 and the anchoring structure 108 may be a different material than that of the linking portion 114. For instance, the stent graft 102 and the anchoring structure 108 could be a metal material, while the linking portion 114 could be fabricated of a polymeric material.

The curvature of the linking portion 114 and/or orientation of the stent graft 108 to the anchoring structure 102 with respect to one another may vary depending on the particular arcuate lumen being treated or a particular patient. In many cases, the linking portion 114 may be resilient, either due to the material used to form the linking portion, the geometry of the linking portion, or both. For example, the reduced cross-section of the linking portion 114 (e.g., relative to that of the stent graft and anchoring structure 108, 102) may make an otherwise straight linking portion sufficiently flexible to conform to an arcuate portion of a lumen.

The size and configuration of the opening 116 may depend on the particular linking portion 114 employed. In addition, the size and configuration of the opening 116 chosen may depend on the number and location of branch lumens to be aligned with the opening. For example, a linking portion 114 comprising a thin or small diameter wire or band would provide a large opening 116 (see e.g., FIG. 10), whereas an opening defined in a braided fabric may be much smaller (see e.g., FIGS. 1 and 8). Furthermore, in one embodiment, the linking portion 114 may include a “loose” fabric that is less densely braided than the stent graft 108 and anchoring structure 102, such that the blood may readily flow through the larger openings 116 defined in the loose fabric. Therefore, at least one opening 116 may be defined in the linking portion 114 and may be located at one or more locations in the linking portion (including up to about the entire circumference of the linking portion).

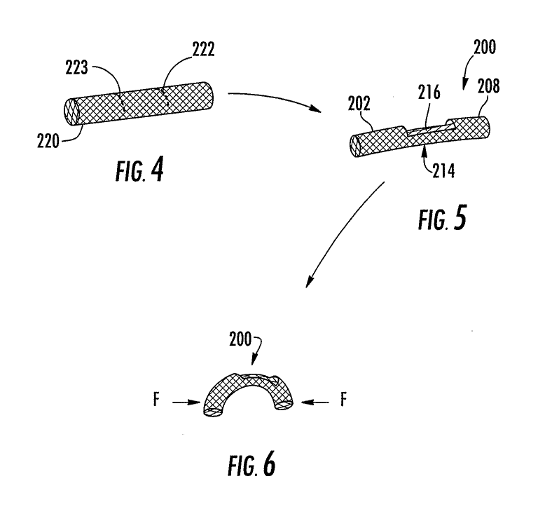

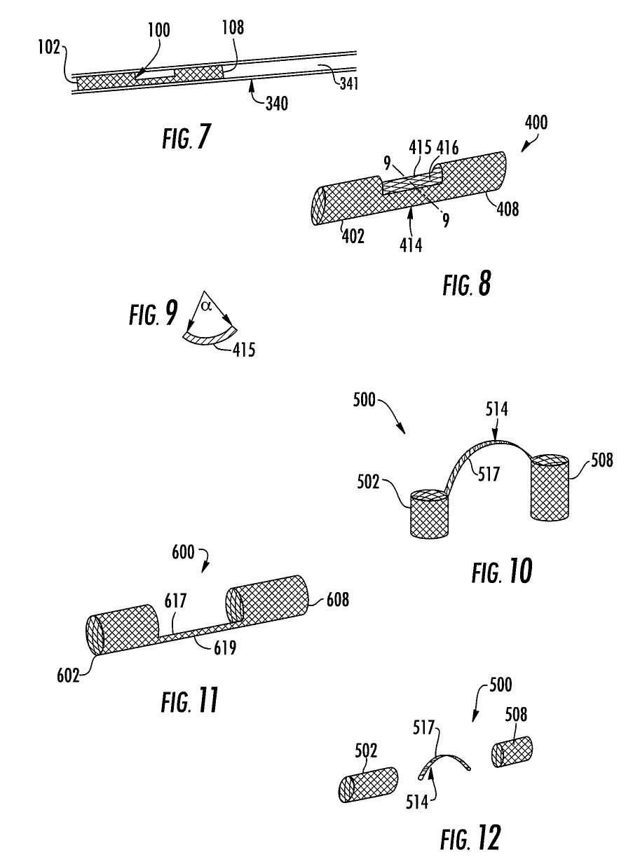

Still referring to FIGS. 1 and 2, the stent graft 108, anchoring structure 102, and linking portion 114 can be integrally formed from a common material. For example, referring now to FIGS. 4-6, a single braided metallic (e.g., Nitinol) tube 220 can be formed by partially cross cutting the tube 220 along sections 222, 223 (e.g., by cutting the wire strands) in order to form a medical device 200 having a linking portion 214 and opening 216 between a first tubular portion 208 and a second tubular portion 202. The linking portion 214 may be formed by axial elongation to reduce its diameter and constraining the linking portion during a heat setting operation to memorize the constrained diameter. During the same heat treatment process, the stent graft 108 and anchoring structure 102 may be heat set in their expanded diameters to memorize their shape. The braided tube 220 may also be heat formed so as to have an arcuate region in the portion that will become the linking portion 214, or can be processed after forming the linking portion 214 such that the linking portion assumes an arcuate configuration, for example, by being forced (e.g., via forces F in FIG. 6) into an arcuate shape and then heat set.

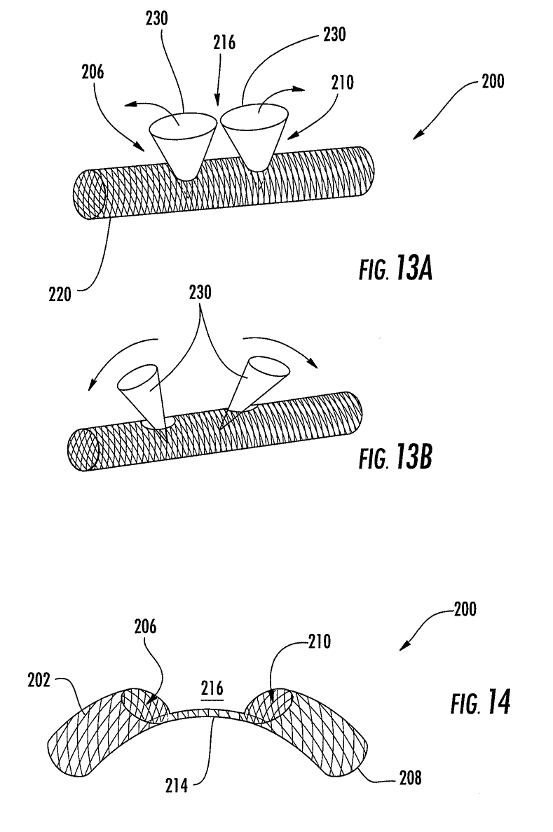

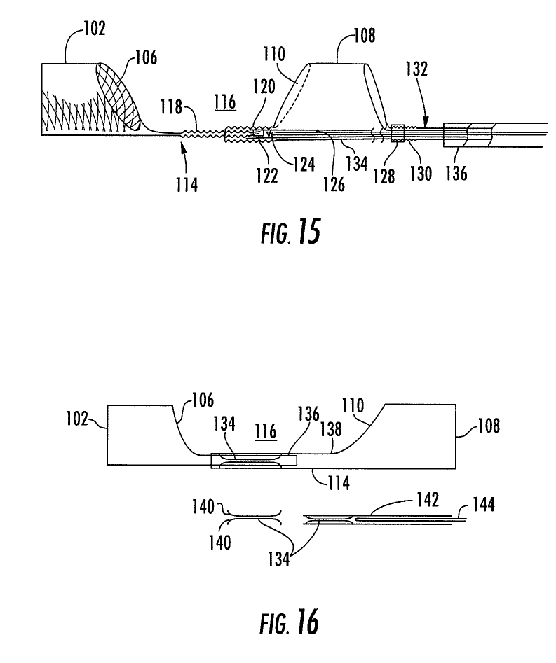

FIGS. 13A-B and 14 illustrate a medical device 200 according to another embodiment of the present invention. The device 200 may be formed from a single length of tubular braided shape memory alloy capable of being heat treated to have a shape transformation temperature below body temperature (e.g., 20-37° C.). Openings 216 can be formed in the tube 220 by pushing a cone shaped probe 230 into the side wall at one location but typically two axial aligned locations along the outer surface to form the openings 206, 210 as shown in FIG. 13B. Once the wires have been displaced by the conical probes 230 sufficiently to establish the desired opening diameter, the braided portion between the two openings 206, 210 can be axially elongated to form the linking portion 214 and opening 216. The process of forming the device 200 will result in loose wires that need to be manually realigned by axial tension from either wire ends while holding the device in the desired final device shape. Once the wires are aligned as desired, the device may be heat set to memorize the desired final shape. The final device shape as shown in FIG. 14 has formed openings 206, 210 at an angle less than perpendicular to the central axis of the tubular portions 202, 208.

Referring to FIGS. 8 and 9, therein is shown a medical device 400 with the stent graft 408 and anchoring structure 402 disposed coaxially with one another according to one embodiment of the present invention. The opening 416 extends between the stent graft 402 and anchoring structure 408 and may be defined, in cross section, by a curved (e.g., circular) sector having an angle a between 0 and 360 degrees. For example, the opening 416 may have an angle a in the range of about 45 to about 225 degrees. The medical device 400 shown in FIGS. 8 and 9 could be resilient structure and thereby conform to an arcuate vessel or heat set in the arcuate configuration as described above.

Referring to FIGS. 10-12, therein are shown medical devices 500, 600 configured in accordance with further exemplary embodiments. The devices 500, 600 include first tubular portions 508, 608 and second tubular portions 502, 602. A linking portion 517, 617 couples the corresponding first and second tubular portions 508, 502, 608, 602. As indicated above, the linking portion 517, 617 can be include a filament, a fiber, a wire, a cord, a cable, a braid, a fabric, and/or a beam. In either case, the thickness of the linking portion 517, 617 can be sufficiently small as compared to its length so as to be relatively flexible. The flexibility of the linking portion 517, 617 may, in some cases, facilitate either delivery of the device 500, 600 to a target site in the body or the ability of the device to conform to the target site once delivered. The device 500 may be fabricated by separately forming the first 508 and second 502 tubular portions, forming the linking portion 517, and coupling the beam to the first and second tubular portions (e.g., with adhesives or laser welding).

In a further embodiment the medical device 600 may be fabricated by laser cutting or acid etching a pattern into a shape memory tube to form the first and second tubular portions 602, 608 (see FIG. 11) by removing most of the circumference of a central portion between the tubular portions to leave a remainder linking portion 614 and corresponding opening 616. Alternatively, the tubular portions 602, 608 may be fabricated as individual components and connected to a separate linking portion 617 similar to that shown in FIG. 12. The tubular portions 602, 608 may be manually expanded to the desired diameter and/or curved to an arcuate preset shape and along with the linking portion 617, heat set in an oven while constrained to the desired final shape to memorize the desired final device shape. The tubular portions 602, 608 may be radially compressed in diameter or elongated for delivery through a catheter to a treatment site within the body. The device may self expand to the memorized shape upon exiting the catheter. Catheter based delivery devices for self expanding stents may be an appropriate means for delivery of the medical device 600. It should be noted that the devices 100, 200, 400, 500, 600 may be sized larger than the vessel diameter by 10-30% to ensure that the device exhibits an anchoring force against the vessel wall. The devices, therefore, may not achieve 100% of their preset shape when exiting a catheter restraint due to vessel resistance to expansion.

Referring again to FIG. 1, in some embodiments, the linking portion 114 may be adjustable in length. For example, the linking portion 114 may include a compressed braid that can be selectively decompressed (and, in some cases, re-compressed) to an extent that is adjustable. Alternatively, the linking portion 114 can include a series of everting links.

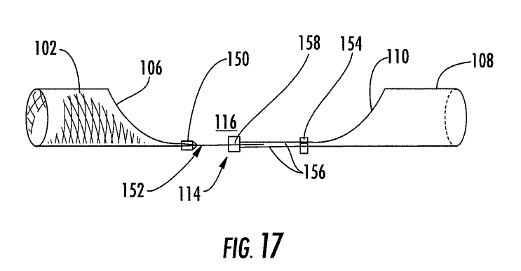

FIG. 15 illustrates one exemplary embodiment for facilitating the length adjustment of the linking portion 114. In particular, FIG. 15 illustrates that the anchoring structure 102 and stent graft 108 may include respective threaded portions 118, 120 configured to engage one another. The anchoring structure 102 may include a threaded connector 122 that is configured to engage a threaded end 124 on a distal delivery device 126. The stent graft 108 may also include a threaded connector 128 that is configured to engage a threaded end 130 on a proximal delivery device 132. The distal delivery device 126 is deliverable through an internal sheath 134 and catheter 136. Thus, both the distal delivery device 126 and internal sheath 134 are configured to be axially displaced through the threaded connector 128 and proximal delivery device 132. The length of the linking portion 114 may be adjusted by threading the threaded portions 118, 120 with respect to one another, which may occur before delivery of the device based on an image of the target site (e.g., using fluoroscopy), or the device could be removed prior to being fully deployed and the length of the linking portion adjusted. When the threaded ends 124, 130 are engaged with respective threaded connectors 122, 128, rotation of the distal 126 and proximal 132 delivery devices results in adjustment of the length of the linking portion 114 as the threaded portions 118, 120 are rotated with respect to one another.

FIG. 16 illustrates another embodiment wherein the length of the linking portion 114 may be adjusted using a locking member 134. More specifically, the locking member 134 may be configured to engage respective free ends 136, 138 of the anchoring structure 102 and stent graft 108. Thus, once the free ends 136, 138 have been axially displaced with respect to one another to achieve a desired length of the linking portion 114, the locking member 134 may engage the free ends together to prevent any further axial displacement. The locking member 134 may include a pair of hook-shaped members 140 that are configured to engage the free ends 136, 138 and may include various materials, such as a metallic material. The locking member 134 may be configured to self expand upon release from a delivery catheter 142, which may be facilitated by a pusher shaft 144, wherein both the delivery catheter and pusher shaft are capable of being axially displaced within the free ends 136, 138 to a desired location prior to release of the locking member. The end of the delivery catheter 142 may include a material capable of resisting puncture by the locking member 134 and facilitate axial displacement of the locking member out of the delivery catheter. For example, the distal end of the delivery catheter 142 may be a metallic material or reinforced sleeve, while the remaining portion of the delivery device may be a flexible, polymeric material. The locking member 134 may be radially constrained for delivery within the delivery catheter 142, and the pusher shaft 144 may be used to push the locking member out of the delivery catheter.

Another embodiment of a medical device having an adjustable linking portion 114 is illustrated in FIG. 17. In this particular embodiment, the anchoring structure 102 includes a clamp 150 and a single wire 152 extending proximally therefrom. Similarly, the stent graft 108 includes a clamp 154 and a pair of wires 156 extending distally therefrom, wherein the pair of wires are configured to extend over the wire 152. Once a desired length of the linking portion 114 is obtained by axially displacing the wire 152 and the pair of wires 156 with respect to one another, the wires 152, 156 may be crimped together with a clamp 158 or using any other suitable techniques for securing the wires together, such as with a set screw.

Referring to FIGS. 1-3 and 7, in order to deliver the medical device 100 to a target site within a lumen having an arcuate portion, such as an aortic arch, the stent graft 108 and the anchoring structure 102 can be constrained from respective expanded configurations (shown in FIG. 1) to a smaller diameter (shown in FIG. 2). For example, where the stent graft 108 and the anchoring structure 102 are formed of a braided metallic fabric, each of the stent graft and the anchoring structure may have a first diameter and may be capable of being collapsed to a second, smaller diameter by being axially elongated.

The constrained device 100 can then be positioned in a delivery catheter 340, which is a catheter that defines an axial bore 341. In this way, the device 100 is maintained in the constrained configuration during delivery by the wall defining the bore 341 of the catheter 340. The catheter 340 and device 100 can then be advanced, for example, over a guidewire, until disposed at the target site (in this case the aortic arch area), where the device 100 can be deployed from the catheter. Once the device 100 has been deployed completely out of the catheter 340, the stent graft 108 and anchoring structure 102 may assume the expanded shape (to the extent permitted by the surrounding vasculature, e.g., the ascending and descending thoracic aorta, respectively) and the linking portion may conform to the arcuate portion of the lumen (e.g., the aortic arch). Further examples of the procedures by which a medical device configured in accordance with exemplary embodiments can be delivered are provided in U.S. Patent Appl. Publ. No. 2006/0253184 filed May 4, 2005, which is hereby incorporated by reference in its entirety.

In some embodiments, the stent graft 108 and anchoring structure 102 may self-expand upon being deployed from the catheter 340 as the constraining forces of the catheter are removed. In other embodiments, the stent graft 108 and anchoring structure 102 may be physically urged into or toward the expanded shape, say, by inflating a balloon located within the stent graft and anchoring structure, or by axially compressing the tube following deployment from the catheter 340.

The location of the medical device 100 may be rotationally dependent on the location of one or more branch lumens extending from the arcuate lumen, such as the AA. Thus, the linking portion 114 may be positioned opposite the openings of the branch lumens, while the opening 116 may be configured to align with the openings of the branch lumens in order to facilitate fluid flow therethrough. In order to aid in the alignment of the medical device within the lumen, the medical device may also comprise one or more radiopaque markers to indicate angular orientation of the device such that the linking portion 114 is located along the inside smallest radius of the arcuate lumen (see e.g., FIG. 3). For example, radiopaque markers could line the linking portion or the openings of the tubular portions adjacent the ostia of the branch lumens, and/or the braid itself could include one or more radiopaque strands so that the medical device is properly positioned and does not block any branch lumens. Radiopaque markers may also facilitate location of the anchoring portion 102 and the stent graft 108 relative to desired target locations. It is further contemplated that the expanded diameter portions of the anchoring portion 102 and the stent graft 108 may be heat set to incorporate a corrugated portion or a sinusoidal wave pattern in the outer surface to increase radial strength as described in pending U.S. patent application Ser. No. 12/181,639, entitled Medical Device including Corrugated Braid and Associated Method. The anchoring portion 102 and/or the stent graft 108 may additionally comprise hooks for engaging the lumen to ensure the device does not migrate.

Embodiments of the present invention may provide several advantages. For example, the medical device is capable of conforming to a variety of arcuate portions within a vessel and is, thus, adaptable for a variety of target sites and patients. The medical device may include a heat set or resilient linking portion that facilitates such adaptability. The linking portion may include an opening that is configured to align with one or more branch vessels extending from the arcuate portion such that the opening reduces blockage in the arcuate portion, such as in the aortic arch. The medical device may also include a stent graft configured to facilitate occlusion at a target site, such as at an aneurysm. Moreover, the medical device may include an anchoring structure in order to facilitate fixation within the vessel and reduce the incidence of migration. Therefore, the medical device is capable of treating target sites within a vessel that may be otherwise difficult to anchor therein or susceptible to blockage of branch vessels when a conventional stent graft is employed.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1-23. (canceled)

24. A medical device for treating a target site within a lumen having an arcuate portion and at least one branch lumen extending therefrom, said medical device comprising:
a first tubular portion comprising a proximal end and a distal end, said first tubular portion formed of a continuous tube between the proximal and distal ends thereof, wherein the first tubular portion is self-expanding;
a second tubular portion comprising a proximal end and a distal end, said second tubular portion formed of a continuous tube between the proximal and distal ends thereof, wherein the second tubular portion is self-expanding; and
a resilient linking portion extending from the distal end of said first tubular portion to the proximal end of said second tubular portion thereby coupling said first and second tubular portions, the resilient linking portion consisting of a shape-memory material and comprising a preset, memorized arcuate configuration extending along, and biased towards, an arcuate axis in a relaxed state and configured to conform to at least a portion of the arcuate portion of the lumen, the resilient linking portion configured to be deflected away from the arcuate axis for delivery to the target site within a catheter and biased such that the resilient linking portion is configured to at least partially return to its preset, memorized arcuate configuration upon being deployed from the catheter at the target site; and
an opening extending from the distal end of said first tubular portion to the proximal end of said second tubular portion, a central axis extending from the proximal end of said first tubular portion, through the opening, and to the distal end of said second tubular portion, wherein a length of the resilient linking portion from the distal end of said first tubular portion to the proximal end of said second tubular portion is less than a length of the opening taken about the central axis from the distal end of said first tubular portion to the proximal end of said second tubular portion, and wherein said opening is configured to align with the at least one branch lumen and facilitate fluid flow between the at least one branch lumen and the arcuate portion of the lumen.

25. The medical device of claim 24, wherein said resilient linking portion includes a structure selected from the group consisting of a filament, a fiber, a wire, a cord, a cable, a braid, a fabric, and a beam.

26. The medical device of claim 24, wherein said first and second tubular portions each comprises at least one layer of a metallic fabric material comprising a plurality of braided strands.

27. The medical device of claim 26, wherein said at least one layer of metallic fabric material comprises a shape memory alloy.

28. The medical device of claim 26, wherein said at least one layer of metallic fabric material comprises a plurality of layers of metallic fabric material, each of the plurality of layers of metallic fabric material comprising a tube formed from a plurality of braided strands.

29. The medical device of claim 26, wherein said first and second tubular portions each includes at least one layer of a metallic fabric material heat set in an expanded heat set configuration.

30. The medical device of claim 24, wherein said first tubular portion, said second tubular portion, and said resilient linking portion are integrally formed from a single tube of shape-memory material.

31. The medical device of claim 30, wherein said first tubular portion, said second tubular portion, and said resilient linking portion are formed from a single braided tube of metallic material.

32. The medical device of claim 24, wherein said first and second tubular portions are heat set in an expanded heat set configuration, and wherein said resilient linking portion is adjustable in length so as to adjust a distance between said heat set first and second tubular portions.

33. The medical device of claim 24, wherein a location of each of said resilient linking portion and said opening within the arcuate portion of the lumen is rotationally dependent on a location of the at least one branch lumen.

34. The medical device of claim 24, wherein the distal end of said first tubular portion and the proximal end of said second tubular portion extend in respective cross-sectional planes, and wherein the cross-sectional planes are spaced apart axially from one another by the resilient linking portion.

35. The medical device of claim 34, wherein a length between the cross-sectional planes along the central axis is greater than a length of the linking portion such that the cross-sectional planes intersect one another and the resilient linking portion.

36. The medical device of claim 34, wherein the cross-sectional planes extend at respective first and second angles less than perpendicular to the central axis, the first angle defined within the first tubular portion between the central axis and the cross-sectional plane at the distal end of the first tubular portion and the second angle defined within the second tubular portion between the central axis and the cross-sectional plane at the proximal end of the second tubular portion.

37. The medical device of claim 34, wherein a majority of the cross-sectional area of the opening at both the distal end of said first tubular portion and the proximal end of said second tubular portion extend within a respective cross-sectional plane of said first and second tubular portions.

38. The medical device of claim 24, wherein the circumference of the resilient linking portion is uniform along its length and between the distal end of said first tubular portion and the proximal end of said second tubular portion.

39. The medical device of claim 24, wherein the opening is entirely open between the distal end of the first tubular portion and the proximal end of the second tubular portion.

40. The medical device of claim 24, wherein said resilient linking portion, in the preset, memorized configuration, defines an arch shape from the distal end of said first tubular portion to the proximal end of said second tubular portion.

41. The medical device of claim 24, wherein said first and second tubular portions are each formed of a tube having continuous inner and outer surfaces defined about its entire circumference.

42. The medical device of claim 24, wherein the linking portion has radius of curvature defined along the arcuate axis in the preset, memorized configuration, and wherein the linking portion is configured to be deflected to a reduced radius of curvature and is biased to return to the radius of curvature in its preset, memorized arcuate configuration.

43. A medical device for treating a target site within a lumen having an arcuate portion and at least one branch lumen extending therefrom, said medical device comprising:
a first tubular portion comprising a proximal end and a distal end, said first tubular portion formed of a continuous tube between the proximal and distal ends thereof, wherein the first tubular portion is self-expandable;
a second tubular portion comprising a proximal end and a distal end, said second tubular portion formed of a continuous tube between the proximal and distal ends thereof, wherein the second tubular portion is self-expandable; and
a resilient linking portion extending from the distal end of said first tubular portion to the proximal end of said second tubular portion thereby coupling said first and second tubular portions, the resilient linking portion comprising a preset, heat set, memorized arcuate configuration configured to conform to at least a portion of the arcuate portion of the lumen, the resilient linking portion configured to at least partially return to its preset, heat set, memorized arcuate configuration upon being deployed at the target site; and
an opening extending from the distal end of said first tubular portion to the proximal end of said second tubular portion, a central axis extending from the proximal end of said first tubular portion, through the opening, and to the distal end of said second tubular portion, wherein a length of the resilient linking from the distal end of said first tubular portion to the proximal end of said second tubular portion is less than a length of the opening taken about the central axis from the distal end of said first tubular portion to the proximal end of said second tubular portion, and wherein said opening is configured to align with the at least one branch lumen and facilitate fluid flow between the at least one branch lumen and the arcuate portion of the lumen.
